(19) United States
(12) Patent Application Publication
(10) Pub. No.: US 2015/0038908 A1
(43) Pub. Date: Feb. 5, 2015

(54) OVER THE NEEDLE CATHETER WITH CURVILINEAR SLIT

(71) Applicant: Luther Needlesafe Products, Inc., Mission Viejo, CA (US)

(72) Inventor: JOSEPH ANTONUCCI, MISSION VIEJO, CA (US)

(73) Assignee: Luther Needlesafe Products, Inc., Mission Viejo, CA (US)

(21) Appl. No.: 13/954,611

(22) Filed: Jul. 30, 2013

(51) Int. Cl. A61M 25/00; A61M 25/06; A61M 5/158

(52) U.S. Cl. CPC A61M 25/0021; A61M 5/158; A61M 25/0075; A61M 25/0606
USPC 604/164.01

(57) ABSTRACT

An over-the-needle catheter having a curvilinear slit formed therein to allow for infusion of fluids through the catheter and into the blood vessel of the patient in the event of blockage of the primary opening of the catheter.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Technical Field

The present disclosure relates generally to an IV catheter, and more specifically to an IV catheter having a curvilinear slit formed adjacent a patient-side end portion of the catheter for providing an auxiliary infusion passageway for use in the event of occlusion/blockage of a primary infusion passageway.

2. Related Art

It is well known in the medical profession that various medical treatments and procedures oftentimes require the insertion of fluid into a patient. Catheters are commonly employed to achieve such infusion of fluid. For instance, catheters are typically used to provide IV access for frequent or continuous injections of medications or fluids for nutritional support. A conventional catheter typically includes a generally inflexible tube having a hard/rigid distal tip. The catheter is typically inserted into a patient's vein using a catheter introduction device, and may remain in the patient for anywhere from several hours to several days.

A drawback commonly encountered with catheter usage, particularly when the catheter has resided within the patient for an extended period of time, is that the end portion of the catheter becomes occluded with blood and may be either completely or partially obstructed, thus inhibiting fluid flow through the catheter. One solution has been to provide catheters with a hard, low friction tip so configured such that subsequent IV infusions may dislodge any occlusions blocking the passage through the catheter. This however presents the additional problem of a blood clot floating freely through the vascular system of the patient, which may result in serious trauma. Accordingly, catheters are typically frequently replaced, on the order of every 2-3 days, as they become occluded with blood.

In view of the foregoing, there is a need in the medical field for an improved catheter that is sized and configured to allow for continuous fluid flow therethough in the event that the end of the catheter becomes occluded with blood. The present invention addresses this need, as will be discussed in more detail below.

### BRIEF SUMMARY

According to an aspect of the invention, there is provided an over-the-needle catheter having a curvilinear slit formed therein to allow for infusion of fluids through the catheter and into the blood vessel of the patient in the event of blockage of the primary opening of the catheter.

In one embodiment, the over-the-needle catheter includes a catheter tube having a proximal (i.e., patient-side) end portion and an opposed distal end portion. The catheter tube further includes an outer surface and an inner surface defining a tube passageway extending between the proximal and distal end portions. A curvilinear slit is formed in the catheter tube adjacent the proximal end portion thereof and extends through the catheter tube from the outer surface to the inner surface and in fluid communication with the tube passageway.

The over-the-needle catheter may include a hardened tip connected to the catheter tube adjacent the proximal end portion thereof. The catheter tube may define a length of 4 inches or less.

The curvilinear slit may define a sinusoidal configuration or an arcuate configuration. The curvilinear slit may additionally circumscribe the catheter tube at least once and define a helical configuration.

The curvilinear slit may be defined by a pair of flaps configured to be transitional between a closed position wherein fluid flow therethrough is inhibited, and an open position wherein fluid flow therethrough is facilitated. The catheter tube may be formed from a resilient material causing the flaps to be biased toward the closed position. A pressure differential between the tube passageway and the outside of the catheter body of about ½ psi to about 5 psi may cause the flaps to transition from the closed position toward the open position.

According to another embodiment, the over-the-needle catheter includes a pair of flaps formed in the catheter tube adjacent the proximal end portion thereof to define an opening through the catheter tube and in communication with the tube passageway, the pair of flaps defining complimentary curvilinear edges.

The presently contemplated embodiments will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is an upper perspective view of a catheter constructed in accordance with an embodiment of the present invention;

FIGS. 2 and 3 are upper perspective views showing insertion of the catheter into a patient;

FIG. 4 is a partial, enlarged, upper perspective view of a proximal end portion of one embodiment of the catheter including a sinusoidal shaped slit extending in a longitudinal direction and in a closed configuration;

FIG. 5 is a partial, enlarged, upper perspective view of the catheter depicted in FIG. 4, wherein the sinusoidal slit is in an open configuration;

FIG. 6 is a partial, enlarged, upper perspective view of a proximal end portion of a second embodiment of the catheter including a sinusoidal shaped slit extending in a circumferential direction, the slit being in a closed configuration; and

FIG. 7 is a partial, enlarged, upper perspective view of a proximal end portion of a third embodiment of the catheter including a helical shaped slit in a closed configuration.

Common reference numerals are used throughout the drawings and the detailed description to indicate the same elements.

### DETAILED DESCRIPTION

The detailed description set forth below in connection with the appended drawings is intended as a description of the presently preferred embodiments of the invention, and is not intended to represent the only form in which the present devices may be developed or utilized. It is to be understood, however, that the same or equivalent functions may be accomplished by different embodiments that are also intended to be encompassed within the spirit and scope of the invention. It is further understood that the use of relational terms such as first, second, and the like are used solely to distinguish one from another entity without necessarily requiring or implying any actual such relationship or order between such entities.

Referring now to the drawings, wherein the showings are for purposes of illustrating preferred embodiments of the present invention, and are not for purposes of limiting the same, there is depicted an over-the-needle catheter 10 specifically configured and adapted to allow for infusion of fluids therethrough despite occlusion over the tip the catheter 10. Along these lines, the catheter 10 includes a curvilinear slit 12 formed therein which functions as an auxiliary port through which fluid may be infused into the patient when the primary opening 14 formed at the tip of the catheter 10 is blocked or occluded.

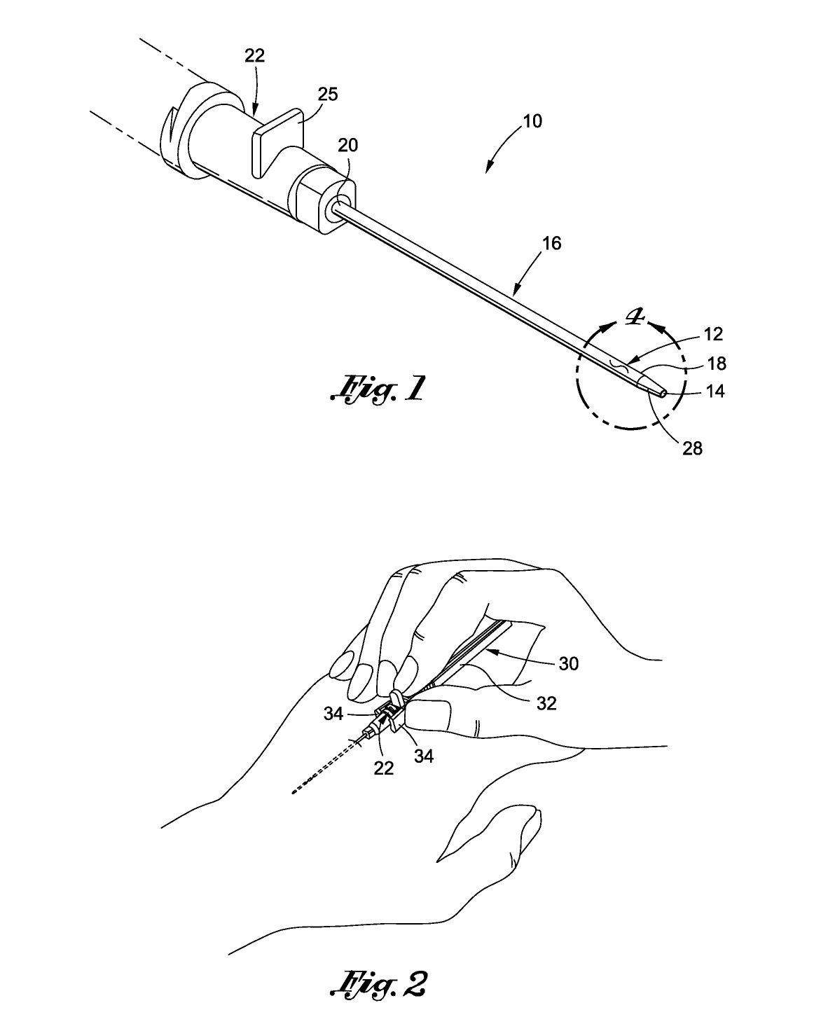

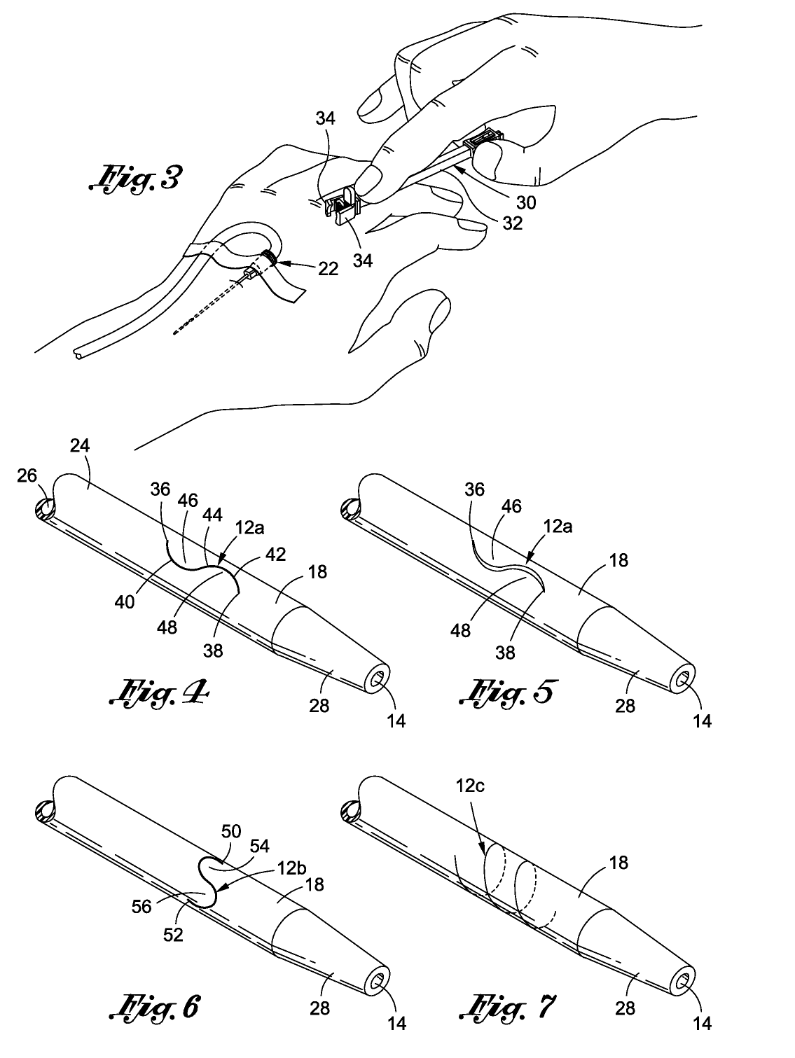

Referring now specifically to FIG. 1, the catheter 10 includes a catheter tube 16 having a proximal end portion 18 insertable into a patient, and an opposing distal end portion 20 coupled to a fitting or hub 22. The catheter tube 16 includes an outer surface 24 (see FIG. 4) and an opposing inner surface 26 (see FIG. 4), wherein the inner surface 26 defines a fluid passageway extending axially along the catheter tube 16 between the proximal end portion 18 and the distal end portion 20.

The catheter tube 16 is preferably formed from a soft, flexible material which allows the catheter 10 to reside within the patient for extended periods of time without causing too much discomfort to the patient. Exemplary materials include a soft polyurethane or thermoplastic material, although other materials known by those skilled in the art may also be used without departing from the spirit and scope of the present invention. The catheter tube 16 may define a length of 4 inches or less.

A hardened tip 28 is coupled to the catheter tube 16 adjacent the proximal end portion 18 thereof and is co-axially aligned therewith. The hardened tip 28 also defines an opening which is in fluid communication with the fluid passageway defined by the catheter tube 16. The hardened tip 28 is configured to provide sufficient rigidity to prevent the catheter tube 16 from collapsing upon insertion and to prevent the catheter tube 16 from traveling axially backwards relative to the insertion needle upon which the catheter tube 16 is disposed during the insertion process. The hardened tip 28 is more readily insertable through the patient's skin and tissue and is more resistant to deformation and may therefore facilitate insertion of the catheter 10 into the patient's vasculature by preventing compaction or axial collapsing of the catheter tube 16 during the insertion process.

The hardened tip 28 is preferably separately formed from the catheter tube 16, and later joined or adhered to the catheter tube 16. However, it is additionally contemplated that the hardened tip 28 may be co-molded with the catheter tube 28 to define an integral structure, wherein the hardened tip 28 may be formed from a separate polymer from that used to form the catheter tube 16. The polymer used to form the hardened tip 28 preferably exhibits an increased hardness relative to the polymer used to form catheter tube 16.

The hub 22 is disposed opposite the hardened tip 28 and is preferable a female Luer lock configured to be selectively connectable to catheter insertion tool 30 for inserting the catheter 10 into the patient, as shown in FIGS. 2 and 3. The hub 22 may include a fin 25 for use in grabbing or manipulating the catheter 10.

The insertion tool 30 shown in FIGS. 2 and 3 is specifically configured and adapted for use with over-the-needle catheters 10, and includes a needle (not shown), a sheath 32, and a pair of arms/jaws 34, which are moveable and configured to capture/release the hub 22 of the catheter 10. When the catheter is 10 engaged with the insertion tool 30, as shown in FIG. 2, the needle is extended through the catheter tube 16 and the hardened tip 28, with the piercing end of the needle exposed so as to facilitate insertion into the patient. The hardened tip 28 preferably rests against a complimentarily-shaped shoulder formed on the needle so as to prevent the catheter 10 from collapsing along the needle during insertion. The hub 22 is additionally captured between the arms 34 to couple the catheter 10 to the insertion tool 30. After the catheter 10 has been placed within the vasculature of the patient, the needle is withdrawn from the catheter 10 and the arms 34 release the hub 22, as shown in FIG. 3. For a more detailed description of the insertion tool 10 or the process of inserting the catheter 10 into the patient using the insertion tool 10, please refer to Applicant's co-pending U.S. application Ser. No. 13/945,728, entitled Low Profile Passive Protector for an I.V. Catheter, the contents of which are expressly incorporated herein by reference.

The catheter 10 additionally includes a curvilinear slit 12 formed in the catheter tube 16 adjacent the proximal end portion 18 thereof. The slit 12 extends through the catheter tube 16 between the outer surface 24 and the inner surface 26 and is in fluid communication with the tube passageway. The slit 12 assures continued fluid flow through the catheter tube 16 in the event the primary opening 14 becomes fully or partially obstructed.

The slit 12 is configured to be transitional between a closed position (see FIG. 4) and an open position (see FIG. 5). The catheter tube 16 preferably is formed from a resilient material which biases the slit 12 toward the closed position. Therefore, during normal operation of the catheter 10, fluid flows through the catheter tube 16 and exits the catheter 10 via the primary opening 14. In the event the primary opening 14 becomes occluded, for example by the buildup of clotted blood over time, the pressure differential across the slit 12 resulting from the inability of the infusion fluid to flow through the primary opening 14 will create an internal fluid pressure which overcomes the biasing force and causes the slit 12 to transition from the close position to the open position, thereby assuring that the infused fluid flows into the patient's vasculature. A pressure differential between the tube passageway and the outside of the catheter tube 16 of about ½ psi to about 5 psi may cause the slit 12 to transition from the closed position toward the open position. In this regard, the slit 12 provides an alternate means for effectuating fluid flow from the catheter tube 16 to the patient. Preferably, the slit 12 is formed adjacent the proximal end portion 18 to ensure that the slit 12 is located within the patient's vasculature when the catheter 10 is positioned within the patient, and to ensure that the slit 12 is in fluid communication with the same anatomical vessel within which the primary opening 14 is disposed.

The slit 12 is preferably formed within the catheter tube 16 so as to define a curvilinear shape, which provides several advantages associated with infusing fluid into the patient's blood vessel. One advantage associated with the curvilinear shape of the slit 12 is that it defines a larger opening through which fluid may flow when the slit 12 is in the open configuration relative to a slit which defines a straight line. The large opening defined by the curvilinear slit 12 is particularly advantageous considering the small dimensions of the catheter tube 16, i.e., the catheter tube 16 defines a small diameter providing limited space for forming the slit 12. Another advantage associated with the curvilinear shape of the slit 12 is that the curved contours of the slit opening tend to create a turbulent fluid flow of the infused fluid, which enhances dissipation of the infused fluid within the blood vessel.

The curvilinear slit 12 can define several different configurations. FIGS. 4-7 depict several different embodiments of a curvilinear slit 12, although those skilled in the art will readily appreciate that the embodiments shown in FIG. 4-7 are exemplary in nature only and are not intended to limit the scope of the present invention.

Referring first to the embodiment depicted in FIGS. 4 and 5, the curvilinear slit 12a defines a sinusoidal configuration. The sinusoidal shaped slit 12a includes a first end 36 and an opposing second end 38. The slit 12a additionally includes an arcuate concave portion 40 adjacent the first end 36 and an arcuate convex portion 42 adjacent the second end 38, wherein the concave portion 40 transitions to the convex portion 42 at an inflection point 44. The first and second ends 36, 38 may be co-axially aligned with each other along a longitudinal axis of the slit, or alternatively, the first and second ends 36, 38 may be offset from a longitudinal axis passing through the inflection point 44.

The sinusoidal curvilinear slit 12a is positioned between a first flap 46 and a second flap 48 integral with the catheter tube 16. The first and second flaps 46, 48 each define complimentary curved edges and are each independently moveable relative to the catheter tube 16 in order to open and close the slit 12a. FIG. 4 shows the slit 12a in the closed position, with the flaps 46, 48 effectively closing the slit opening to prevent fluid flow therethrough. FIG. 5 shows the slit 12a in an open position, wherein the flaps 46, 48 are spaced from each other to allow fluid to flow through the slit opening.

FIG. 6 shows another embodiment of the curvilinear slit 12b, wherein the slit 12b is similar to the slit 12a shown in FIGS. 4 and 5, although the slit 12b in FIG. 6 is oriented in a slightly different orientation relative to the slit 12a in FIGS. 4 and 5. In particular, the slit in FIG. 6 extends in a circumferential direction, whereas the slit 12a in FIGS. 4 and 5 extends in a generally axial direction. Along these lines, the slit 12b includes a first end 50 and a second end 52, wherein the first and second ends 50, 52 preferably reside on a common circumferential axis, although it is understood that the ends 50, 52 may be offset from the circumferential axis.

The sinusoidal curvilinear slit 12b is positioned between a first flap 54 and a second flap 56 integral with the catheter tube 16. The first and second flaps 54, 56 each define complimentary curved edges and are each independently moveable relative to the catheter tube 16 in order to open and close the slit 12b.

FIG. 7 shows yet another embodiment of a curvilinear slit 12c formed in a helical configuration. In this regard, the helical slit 12c circumscribes the catheter tube 16 to form the helical shaped slit 12c. In the exemplary embodiment, the helical slit 12c circumscribes the catheter tube 16 approximately twice, although other embodiments of the helical slit 12c may be shorter, and thus, circumscribe the catheter tube 16 fewer than two times, or longer, and thus, circumscribe the catheter tube 16 more than two times. The advantage of forming the slit 12c into a helix is that the infused fluid may flow radially outward from the catheter tube 16 in 360 degrees.

In use, the catheter 10 is inserted into the patient's vasculature using the insertion tool 30 or similar means known by those skilled in the art. Once the catheter 10 has been properly placed in the patient's vasculature, an infusion fluid may be delivered through the hub 22 of the catheter and into the patient's vasculature. During normal operation, the fluid will flow through the catheter 10 and enter the patient's blood stream via the primary opening 14 formed at the proximal end of the catheter 10. As discussed above, during normal operation, the fluid pressure inside of the catheter 10 is smaller than the biasing force, which results in the slit 12 being in the closed configuration. However, in the event the primary opening 14 because blocked or occluded, the pressure inside the catheter 10 will build due to the inability of the infusion fluid to flow through the primary aperture 14. Once the pressure inside the catheter 10 exceeds the biasing force, the slit 12 transitions from the closed position to the open position, thereby allowing the infusion fluid to flow through the slit opening and into the patient's vasculature. In this regard, the slit 12 provides an auxiliary opening through which the infusion fluid may flow, which allows the catheter 10 to remain within the patient's vasculature for extended periods of time, i.e., the catheter 10 does not require removal immediately upon occlusion of the primary opening 14.

The particulars shown herein are by way of example and for purposes of illustrative discussion of the embodiments of the present invention only and are presented in the cause of providing what is believed to be the most useful and readily understood description of the principles and conceptual aspects. In this regard, no attempt is made to show more details than is necessary for a fundamental understanding of the disclosure, the description taken with the drawings making apparent to those skilled in the art how the several forms of the presently disclosed invention may be embodied in practice.

What is claimed is:

1. An over-the-needle catheter for introduction into a blood vessel of a patient, the over-the-needle catheter comprising:
a catheter tube having:
a proximal end portion;
an opposed distal end portion; and
an outer surface and an inner surface defining a tube passageway extending between the proximal and distal end portions; and
a curvilinear slit formed in the catheter tube adjacent the proximal end portion thereof and extending through the catheter tube from the outer surface to the inner surface and in fluid communication with the tube passageway.

2. The over-the-needle catheter recited in claim 1, further comprising a hardened tip connected to the catheter tube adjacent the proximal end portion thereof.

3. The over-the-needle catheter recited in claim 1, wherein the curvilinear slit defines a sinusoidal configuration.

4. The over-the-needle catheter recited in claim 1, wherein the curvilinear slit defines an arcuate configuration.

5. The over-the-needle catheter recited in claim 1, wherein the curvilinear slit circumscribes the catheter tube at least once.

6. The over-the-needle catheter recited in claim 5, wherein the curvilinear slit defines a helical configuration.

7. The over-the-needle catheter recited in claim 1, wherein the curvilinear slit is defined by a pair of flaps configured to be transitional between a closed position wherein fluid flow therethrough is inhibited, and an open position wherein fluid flow therethrough is facilitated.

8. The over-the-needle catheter recited in claim 7, wherein the catheter tube is formed from a resilient material causing the flaps to be biased toward the closed position.

9. The over-the-needle catheter recited in claim 7, wherein a pressure differential between the tube passageway and the outside of the catheter body of about ½ psi to about 5 psi causes the flaps to transition from the closed position toward the open position.

10. The over-the-needle catheter recited in claim 1, wherein the catheter tube defines a length of 4 inches or less.

11. An over-the-needle catheter for introduction into a blood vessel of a patient, the over-the-needle catheter comprising:
a catheter tube having:
a proximal end portion and an opposed distal end portion; and
an outer surface and an inner surface defining a tube passageway extending between the proximal and distal end portions; and
a pair of flaps formed in the catheter tube adjacent the proximal end portion thereof to define an opening through the catheter tube and in communication with the tube passageway, the pair of flaps defining complimentary curvilinear edges.

12. The over-the-needle catheter recited in claim 11, further comprising a hardened tip connected to the catheter tube adjacent the proximal end portion thereof.

13. The over-the-needle catheter recited in claim 11, wherein the curvilinear edges define a sinusoidal configuration.

14. The over-the-needle catheter recited in claim 11, wherein the curvilinear edges define an arcuate configuration.

15. The over-the-needle catheter recited in claim 11, wherein the curvilinear edges circumscribe the catheter tube at least once.

16. The over-the-needle catheter recited in claim 15, wherein the curvilinear edges define a helical configuration.

17. The over-the-needle catheter recited in claim 11, wherein the pair of flaps are configured to be transitional between a closed position wherein fluid flow through the opening is inhibited, and an open position wherein fluid flow through the opening is facilitated.

18. The over-the-needle catheter recited in claim 17, wherein the catheter tube is formed from a resilient material causing the flaps to be biased toward the closed position.

19. The over-the-needle catheter recited in claim 17, wherein a pressure differential between the tube passageway and the outside of the catheter body of about ½ psi to about 5 psi causes the flaps to transition from the closed position toward the open position.

20. The over-the-needle catheter recited in claim 11, wherein the catheter tube defines a length of 4 inches or less.
